(19) United States
(12) Patent Application Publication
(10) Pub. No.: US 2024/0081634 A1
(43) Pub. Date: Mar. 14, 2024

(54) DENTAL RETRACTOR FOR AUTONOMOUS IMAGING, WITH MOUTH CLOSED OR OPEN

(71) Applicant: DENTAL MONITORING, Paris (FR)

(72) Inventors: Thomas PELLISSARD, MAISONS-ALFORT (FR); Cédric LANCON, VILLERS FARLAY (FR); Louis-Charles ROISIN, PARIS (FR)

(21) Appl. No.: 18/271,338

(22) PCT Filed: Jan. 11, 2022
(86) PCT No.: PCT/EP2022/050463

§ 371 Date: Jul. 7, 2023

(30) Foreign Application Priority Data
Jan. 12, 2021 (FR) FR2100236

(51) Int. Cl. A61B 1/32; A61B 1/24

(52) U.S. Cl. CPC A61B 1/32; A61B 1/24

(57) ABSTRACT

A dental retractor including: right, left, upper and lower flanges extending around a retractor opening and intended to rest, in a service position, on the right labial commissure, the left labial commissure, the central part of the upper lip and the central part of the lower lip respectively, of a user. The retractor including: a structure on which the flanges are attached. The retractor including: right and left handles rigidly attached to the right and left ends of the structure respectively, and/or to the right and left flanges respectively, shaped so as to extend outside the mouth of the user in the service position. The structure and/or the attachment of the flanges on the structure is/are shaped so that the horizontal closing force required to bring the right flange and the left flange 10 mm closer together is greater than 5 N.

### TECHNICAL FIELD

The present invention concerns the field of dental image acquisition and more particularly an acquisition kit including a dental retractor facilitating acquisition of dental images, in particular for the execution of a method as described in the international application PCT/EP2015/074896.

### PRIOR ART

PCT/EP2015/074896 describes an acquisition kit enabling acquisition of photos of the teeth of a user. That acquisition kit includes:
a dental retractor intended to be placed in the mouth of the user,
a mobile telephone including a camera.

The dental retractor has right-hand and left-hand flanges known “troughs”, that can be moved elastically closer together to facilitate positioning the dental retractor in the mouth of the user in the service position. The absence of upper and lower flanges advantageously enables the user to open or close their mouth, which is particularly useful in the applications described in PCT/EP2015/074896. However, some teeth, and in particular the incisors, remain partially concealed by the lips.

U.S. Pat. No. 6,923,761 and WO2006014371 describe a dental retractor intended for professional use in the context of surgical operations. That retractor has the advantage of retracting the lips laterally but also vertically, which makes it possible to expose the incisors. However, it is very flexible in order to facilitate placing it in the service position. It is therefore deformed when, in the service position, it is pulled toward the right or the left of the user. This deformability therefore makes it impossible to push back the lips to take lateral or occlusal views, in particular of the teeth at the back of the mouth.

US 2004/0209225 describes a dental retractor intended to be entirely positioned in the mouth of the user. That retractor, configured for professional use, enables the practitioner to access different zones of the mouth of the user. The dental retractor being entirely positioned inside the mouth of the user, it cannot be easily manipulated, however. In particular, it is difficult to pull the retractor toward the right or toward the left without concealing some of the teeth. Moreover, this retractor is rigid in order to maintain the access to the interior of the mouth. In particular, this rigidity prevents closing the mouth for the acquisition of mouth closed photos.

Finally, placing in the service position necessitates contact of the mouth of the user with the fingers of the person manipulating the dental retractor. It therefore requires perfect hygiene conditions. This dental retractor is therefore reserved exclusively for professional use. It cannot be handed to the user for the latter to install it for themselves in the service position in their mouth, in particular to take images using their mobile telephone.

Thus there exists a requirement for a dental retractor:
that can be easily manipulated and placed in the service position in the mouth of the user by the user himself or herself,
that then, in the service position, enables easy acquisition of images of the teeth at the back of the mouth, in particular in occlusal or lateral views, and closed mouth images.

An objective of the present invention is to address this requirement at least in part.

### STATEMENT OF INVENTION

Retractor According to the Invention

The invention proposes a dental retractor including:
right-hand, left-hand, upper and lower flanges extending around a retractor opening and intended, in the service position, to bear on the right-hand labial commissure, the left-hand labial commissure, the central part of the upper lip and the central part of the lower lip of a user, respectively,
a structure to which said flanges are fixed, and
preferably, a handle shaped in such a manner as to extend outside the mouth of the user in the service position.

The presence of the four flanges is considered indispensable to retract the lips effectively, whilst allowing deformation of the dental retractor, in particular by twisting it.

The handle allows manipulation of the dental retractor and in particular deformation thereof to be placed in the service position by the user himself or herself. This kind of dental retractor is therefore well suited to the user himself or herself taking dental photos in an autonomous manner.

In accordance with a first principal aspect of the invention the structure and/or the fixing of the flanges onto the structure is (are) shaped in such a manner that the “horizontal” moving closer force necessary to move the right-hand flange and the left-hand flange 10 mm closer together, that is to say the minimum force to be exerted to obtain such moving closer movement, is greater than 5 N, preferably greater than or equal to 6 N, preferably greater than or equal to 8 N, preferably greater than or equal to 10 N.

A moving closer force between two flanges is a traction force exerted parallel to the plane of the retractor on the free end of one of the two flanges to move it elastically closer to the free end of the other flange, which is kept immobile. In other words, the moving closer force is exerted on the free end of the first flange, the free end of the second flange being kept immobile (the dental retractor being retained only by the free end of the second flange). The free end of the second flange is therefore subjected to a reaction force along the same axis, of the same amplitude, but in the opposite direction to the moving closer force.

When the retractor is symmetrical with respect to a vertical median plane passing through the middle of the upper and lower flanges the horizontal moving closer force moving the right-hand flange and the left-hand flange closer by 10 mm is therefore going to produce a 5 mm moving closer movement of each of the free ends of the right-hand and left-hand flanges toward said median plane.

Trials have shown that with a retractor of the type described in U.S. Pat. No. 6,923,761 and WO2006014371 cited in the preamble, marketed by the company YDM and represented in FIG. 17, a horizontal moving closer force of 2.2 N suffices to move 10 mm closer the free ends of the right-hand and left-hand flanges. The retractor tested included a tongue support physically connecting the right-hand and left-hand flanges, but in the absence of any tongue support said horizontal moving closer force is even lower.

As will become more clearly apparent in the remainder of the description, a dental retractor in accordance with the first principal aspect of the invention therefore has a “horizontal” rigidity that enables it to push the lips back effectively when the retractor is pulled toward the right or toward the left of the user and thus facilitates acquisition of images of the teeth at the back of the mouth.

It is therefore well suited to the acquisition of images by the user in an autonomous manner, that is to say without the assistance of a dental care professional, in particular using their mobile telephone.

In accordance with a second principal aspect of the invention the structure and/or the fixing of the flanges onto the structure is/are shaped in such a manner that the “vertical” moving closer force necessary to move 10 mm closer the upper flange and the lower flange is less than the “horizontal” moving closer force necessary to move 10 mm closer the right-hand flange and the left-hand flange.

As will become more clearly apparent in the remainder of the description, a dental retractor in accordance with the second principal aspect of the invention therefore has a vertical flexibility that facilitates moving closer together the upper and lower flanges and therefore the acquisition of photos in mouth open position and mouth closed position, whilst keeping the incisors exposed. A dental retractor according to the invention also facilitates the acquisition of photographs in occlusal and lateral views.

The vertical moving closer movement takes account of the deformation of the structure but also of any mobility of the upper flange and/or of the lower flange relative to the structure, preferably by pivoting.

The upper and lower flanges are preferably fixed to the structure of the retractor by junctions allowing vertical elastic movement of the free ends of said flanges relative to said structure. This “vertical mobility” of said free ends relative to the structure therefore enables the upper and lower lips of the user to move closer together when the user closes their mouth, even if the structure remains rigid. It greatly facilitates this closing.

The amplitude of said vertical elastic movement is preferably greater than 5 mm, preferably greater than 10 mm and/or less than 20 mm.

Said junctions preferably allow said elastic movement because of the effect of a force less than 10 N and greater than 0.5 N exerted on the upper free end of the upper flange or on the lower free end of the lower flange.

Said junctions are preferably nevertheless sufficiently rigid to maintain the front teeth exposed, preferably substantially totally exposed in the mouth closed position, preferably in any position of the mouth between the mouth totally open position and the mouth closed position. The boundary between the upper lip and the teeth of the upper dental arch, as observed through the retractor opening, preferably remains substantially immobile relative to said teeth upon closing the mouth. The boundary between the lower lip and the teeth of the lower dental arch, as observed through the retractor opening, preferably remains substantially immobile relative to said teeth during closing of the mouth. The upper and lower flanges are therefore able to accompany the movement of closing the mouth.

The retractor therefore makes it possible to obtain mouth open and mouth closed images whilst facilitating and improving the acquisition of images in lateral and occlusal views.

In accordance with a third principal aspect of the invention compatible with the other principal aspects the invention proposes a retractor at least in part, preferably completely, gray in color.

The color of the retractor preferably includes, in the RGB (Red, Green, Blue) code, substantially the same value for red, green and blue, the maximum difference between the values for red, green and blue preferably being less than 10, preferably less than 5, preferably substantially zero. These values are preferably between 50 and 210 inclusive, preferably between 70 and 190 inclusive, more preferably between 90 and 170 inclusive, preferably between 140 and 160 inclusive. For example, the retractor may have a color close to (120,120,120), or (150,150,150).

Surprisingly, the inventors have discovered that analysis of the color of the teeth in realistic color photos is facilitated and improved. A dental retractor of this kind therefore enables acceleration of the processing of the photos for a colorimetric analysis.

Without being restricted by this theory, the inventors explain this result by virtue of the fact that the color gray is a neutral color, which does not degrade the colors during acquisition. In fact, the settings conventionally used for acquisition devices, in particular personal acquisition devices such as mobile telephones or tablets, can modify the colors in the photo, in particular by adjusting the white balance.

The retractor is preferably gray in color at least in the region or regions liable to be visible in the images acquired in the service position, in particular when the image acquisition device, in particular a mobile telephone, is attached to the retractor by means of a support. A dental retractor in accordance with the first and/or the second and/or the third principal aspect(s) of the invention may have one or more of the following optional features:
the dental retractor is configured in such a manner as to be entirely on the outside of the teeth in the service position, in such a manner as to allow complete occlusion;
the dental retractor is configured in such a manner as to leave entirely unobstructed the view through the retractor opening in the service position, and preferably has no tongue support;
the dental retractor includes a handle extending outside the mouth of the user in the service position;
the handle takes the form of a tab, preferably oriented toward the center of the retractor opening;
the handle is shaped to be moved away from the exterior surface of the cheeks of the user in the service position;
the handle is fixed to the right-hand or left-hand end of the structure and/or one of the right-hand and left-hand flanges;
the dental retractor includes said right-hand and left-hand handles rigidly fixed to the right-hand and left-hand ends of the structure, respectively, and/or to the right-hand and left-hand flanges, respectively;
the right-hand and left-hand handles are shaped for positioning the dental retractor in said service position with no contact with the mouth of the user, that is to say allowing placing of the dental retractor in the service position without contact of the fingers manipulating the dental retractor with the mouth of the user;
the right-hand and left-hand handles, preferably in the form of tabs, are arranged in such a manner as to extend radially to the outside of the retractor opening;
each flange has a length, as measured along said flange, greater than 1 cm, preferably greater than 2 cm and/or less than 15 cm, preferably less than 12 cm, preferably less than 8 cm;
the ratio of said horizontal moving closer force to said vertical moving closer force is greater than 2, preferably greater than 4, preferably greater than 4, preferably greater than 5, preferably greater than 6, preferably greater than 7, and/or less than 15, preferably less than 12, preferably less than 10;
said horizontal moving closer force is less than 20 N, than 15 N, and/or greater than 5 N, preferably greater than or equal to 6 N, preferably greater than or equal to 8 N, preferably greater than or equal to 10 N;
said vertical moving closer force is less than 8 N, 6 N, 4 N, 3 N, 2 N, preferably less than or equal to 1.5 N, and/or greater than 0.5 N, preferably greater than 1 N;
the structure is shaped in such a manner that deformation of the structure leading to movement of the right-hand and left-hand flanges relative to one another causes movement of the upper and lower flanges relative to one another, said right-hand and left-hand flanges preferably moving closer when the upper and lower flanges are moved apart, and vice versa;
the structure is shaped in such a manner that deformation of the structure leading to movement of the points for fixing said upper and lower flanges to one another leads to movement of the right-hand and left-hand flanges relative to one another, said fixing points preferably moving closer when the right-hand and left-hand flanges move apart, and vice versa;
the structure includes at least one arch, the right-hand and left-hand flanges being fixed, preferably rigidly, to right-hand and left-hand ends of said arch, respectively, so that said arch elastically opposes movement closer together of the right-hand and left-hand flanges;
the retractor includes two of said arches, known as upper and lower arches, preferably coplanar, preferably with the same general shape, preferably symmetrical, to which are fixed the upper and lower flanges, respectively;
the upper and lower arches join at their ends in such a manner that the structure has an overall shape similar to the contour of a rugby ball;
the upper and lower arches are configured to be moved 10 mm closer together by the action of a vertical force moving said arches closer together less than 15 N, 10 N, 8 N, preferably less than or equal to 6 N, and/or greater than 3 N, preferably greater than 4 N, the vertical moving closer force between the two arches being a traction force exerted parallel to the plane of the retractor, at the middle of one of said arches, to move it elastically closer to the middle of the other arch, which is kept immobile;
the upper and lower arches are configured to move 10 mm closer because of the action of a horizontal force moving said arches closer together less than 20 N, than 15 N, and/or greater than 5 N, preferably greater than or equal to 6 N, preferably greater than or equal to 8 N, preferably greater than or equal to 10 N, the horizontal moving closer force between the two arches being a traction force exerted horizontally and parallel to the plane of the retractor on a junction between said arches to move it elastically closer to the other junction between said arches, which is kept immobile;
more generally, the structure is compressible horizontally by 10 mm only because of the action of a horizontal force F_{H} moving the two lateral ends (right-hand and left-hand ends) of the structure closer together greater than 5 N, preferably greater than or equal to 6 N, preferably greater than or equal to 8 N, preferably greater than or equal to 10 N;
the ratio of said horizontal force moving the arches closer together to said vertical force moving the arches closer together is greater than 1, preferably greater than 1.5, preferably greater than 2, preferably greater than 2.3, and/or less than 5, preferably less than 3, preferably less than 2.5;
the upper flange and/or the lower flange is (are) mounted to pivot elastically on the upper and lower arches, respectively, preferably about horizontal axes in the service position, said pivoting of the upper flange and/or of the lower flange allowing vertical movement of the free end of the upper flange and/or of the lower flange, respectively, relative to the upper arch and/or the lower arch, respectively, by a distance of 10 mm because of the effect of a force less than 10 N, 5N, 3 N, 2 N, preferably less than or equal to 1.5 N, and/or greater than 0.5 N, preferably greater than 1 N;
the upper and/or lower flange is (are) made in one piece with said upper or lower arch, respectively;
the structure is shaped in such a manner as to extend radially outside the mouth of the user in the service position;
the upper and lower arches are arranged in such a manner as to extend outside the mouth of the user in the service position;
the upper and lower arches are arranged in such a manner as to extend radially outward from the retractor opening in the service position, that is to say when the retractor is observed along the axis of said opening, said opening appearing to be included in the surface defined by said arches;
the upper flange and/or the lower flange include(s) right-hand and left-hand fins configured to come to abut on the right-hand and left-hand flanges, respectively, in such a manner as to block movement apart of the upper and lower flanges during movement closer together of the right-hand and left-hand flanges;
at any point on one of said upper and lower arches the slope of the arch is greater than or equal to 0° and less than or equal to 60°, preferably less than 50°, preferably less than 45°, preferably less than 40°, the slope being the absolute value of the smallest angle formed by a tangent to said arch at said point with a horizontal plane in the service position, that is to say with a plane perpendicular to the general plane of the retractor and containing the centers of the right-hand and left-hand flanges;
the maximum slope of said arch along said arch is greater than or equal to 20°, preferably greater than 30°, and/or less than or equal to 60°, preferably less than 50°, preferably less than 45°, preferably less than 40°;
the slope of said arch increases continuously, preferably in a regular manner, from the middle of said arch to either one of its two ends;
the upper and/or lower flange(s) include(s) a notch to receive a labial brake;
the notch has a rounded, rectangular, square or triangular shape;
the upper flange and the lower flange each include a notch, the notch of the upper flange and the notch of the lower flange preferably being identical;
the notch has a width greater than 3 mm, 5 mm, 8 mm, 10 mm, the width being measured at the opening of said notch.

Kit According to the Invention

The invention further proposes a kit including:
a dental retractor, preferably in accordance with the first and/or the second and/or the third principal aspect of the invention, and
a retractor support, selectively independent of the retractor, in a disassembled position, or fixed to the retractor, in an assembled position.

In accordance with a fourth principal aspect of the invention, in an assembled position the fixing of the support onto the retractor is configured to impose a distance between the right-hand and left-hand flanges.

As will become more clearly apparent in the remainder of the description, assembling the retractor support and the retractor enables said distance between the right-hand and left-hand flanges to be fixed, and therefore further stiffening the structure of the retractor. The retractor support acts like a locking key that fixes the configuration of the structure of the retractor. In the assembled position the structure of the retractor can therefore be described as “locked”.

Locking the structure of the retractor facilitates the transmission of force to the lips when the retractor is moved laterally or vertically relative to the user in the service position. The lips are therefore pushed back effectively, which in particular facilitates the taking of lateral and occlusal views for the acquisition of photos of the teeth at the back of the mouth.

Locking the structure of the retractor also makes it possible to improve the exposure of the front teeth (incisors, canines), in particular when the user has their mouth open. In a preferred embodiment the free ends of the upper and lower flanges may nevertheless move elastically closer together, including in the assembled position of the retractor. The user can advantageously close their mouth without being too much impeded by the rigidity of the structure of the retractor. The elasticity with which said free ends are moved closer together nevertheless makes it possible to maintain exposure of the teeth from in front.

In the disassembled position the deformability of the structure, that is to say its capacity to be deformed by hand by the user, facilitates in particular the placing of the dental retractor in the service position by the user, in particular thanks to twisting and/or bending of the dental retractor enabling the retractor to be inserted in the mouth of the user.

The structure is preferably configured in such a manner that, in the disassembled position, the right-hand and left-hand flanges can be moved elastically closer together and the upper and lower flanges can be moved elastically closer together. Placement in the service position in the mouth of the user is further facilitated by this.

The retractor support may preferably be fixed to the retractor when the retractor is in the service position. It preferably extends outside the mouth in the service position.

The rigidity of the structure before locking advantageously enables the attachment parts of the retractor to be moved apart by substantially the same distance as the corresponding retractor support attachment parts. This advantageously greatly simplifies assembling the retractor onto the support.

In accordance with a fifth principal aspect of the invention compatible with the other principal aspects the kit is noteworthy in that, in the assembled position, each retractor attachment part is pressed onto a corresponding support attachment part, preferably attracted magnetically by a corresponding support attachment part, along an attachment axis, and in that said retractor and support attachment parts are configured to block by abutment movement, preferably any movement, in a plane perpendicular to the attachment axis.

The retractor and support attachment parts are preferably configured in order, in the service position, to block by abutment at least movement of the support relative to the retractor toward the right of the user, toward the left of the user, upward or downward, in particular in such a manner that in the service position, when the user moves the retractor toward their right or toward their left by means of the support, the support and the retractor remain attached to one another.

As will become more clearly apparent in the remainder of the description assembling the retractor support and the retractor can therefore and advantageously be simple to carry out and reliable. In particular, with magnetic attachments, it suffices to move each retractor attachment part closer to the corresponding support attachment part to ensure pressing together thereof in the direction of magnetic attraction. This pressing together leads to placement of the abutment in its locking position, which thereafter prevents detachment by traction in a plane perpendicular to that direction.

A kit in accordance with the fourth and/or the fifth principal aspect of the invention may have one or more of the following optional features:
the retractor includes retractor attachment parts and said retractor support includes support attachment parts disposed in such a manner as to cooperate, preferably magnetically, with said retractor attachment parts in such a manner as to fix said retractor onto said retractor support removably, optionally rigidly;
the retractor support is rigid between the support attachment parts so that after fixing the retractor onto the retractor support said retractor attachment parts cannot be moved farther apart or closer together;
at least right-hand and left-hand retractor attachment parts are rigidly fastened to the right-hand and left-hand flanges, respectively;
the retractor attachment parts are fixed to the junctions between the lower and upper arches;
the retractor and the retractor support are fixed to one another in the assembled position by one or more clips, self-grip tapes, for example of Velcro® type, clamping jaws, screws, magnets, complementarity of shape between the retractor support and the retractor, elastic attachment means, for example an elastic band, self-adhesive tapes, hooks or suckers;
the kit further includes an image acquisition device, preferably a mobile telephone, fixed to the retractor support, preferably removably, in such a manner as to acquire images representing at least partly the retractor opening.

A kit according to the invention preferably includes a dental retractor according to the first and/or second and/or third principal aspect.

### METHOD ACCORDING TO THE INVENTION

The invention equally concerns a method of acquiring dental images by means of a dental retractor or a kit according to the invention, including the following steps:
a) placing the dental retractor in the service position, in the disassembled position when the method is executed using a kit according to the invention,
b) if the method is executed by means of a kit according to the invention, after step a), fixing the retractor support to the dental retractor so that the dental retractor is in the assembled position,
c) moving the dental retractor, preferably by causing the dental retractor to perform a movement in rotation around the user, toward the right and/or toward the left of the user, so as to render at least one molar visible through the retractor opening, preferably in such a manner that the retractor opening X substantially faces said molar, and/or closing and/or opening the mouth of the user so as to render teeth visible through the retractor opening in the mouth closed position or in the mouth open position, respectively;
d) preferably, acquiring by means of an image acquisition device at least one image representing said molar and/or representing teeth in the mouth closed or the mouth open position.

Step a) includes in particular deformation of the dental retractor by exerting a force on the right-hand, left-hand, upper and/or lower flanges so as to modify the retractor opening and to facilitate insertion of the retractor into the mouth of the user.

Step a) preferably includes twisting the dental retractor about a horizontal axis and/or bending the dental retractor about a vertical axis. Twisting or bending the structure therefore advantageously enables easy introduction of the dental retractor into the mouth of the user, despite the horizontal rigidity.

The presence of right-hand and left-hand fins on the upper and/or lower flange advantageously makes it possible to limit the vertical separation of said upper flange and said lower flange when the right-hand and left-hand flanges are moved closer to position the dental retractor in the service position.

Before step d), preferably before step c), preferably before step b), preferably even before step a), the image acquisition device is fixed to the retractor support so that the lens of the image acquisition device faces the retractor opening. This position therefore enables the image acquisition device to acquire an image representing the retractor opening and therefore images of the interior of the mouth in the service position.

In a particular embodiment the image acquisition device cannot be separated from the retractor support. For example, the retractor support may include an integrated camera or video camera the lens of which faces the retractor opening.

The image acquisition device is preferably fixed to the retractor support, preferably removably, so that its optical axis always crosses the retractor opening whatever the position of the image acquisition device on the retractor support.

In step c) the movement of the dental retractor may be effected thanks to movement of the retractor support when the dental retractor is fixed to the retractor support.

Steps c) and d) are preferably repeated several times, for example by effecting a first rotation to the right in order to acquire a photo of the right-hand molars and then by effecting a second rotation to the left in order to acquire a photo of the left-hand molars.

The dental retractor can equally be moved upward and/or downward in order to acquire images of occlusal views. The user can alternatively or additionally acquire one or more mouth open photos and then mouth closed photos or vice versa.

A step, preferably all the steps, may be effected by the user himself or herself or someone close to them.

Visual and/or audio instructions may be generated. In particular, instructions may tell the user how to position the dental retractor in the service position, in particular how to deform the dental retractor to facilitate positioning it in the service position, how to fix the retractor support to the dental retractor and/or the image acquisition device to the retractor support for use of a kit in accordance with the invention, how many images they must acquire and/or the movements to be effected to acquire different views. The instructions may be emitted by the image acquisition device.

The invention also concerns:
a computer program or “application” and in particular a dedicated application for mobile telephones containing program code instructions for guiding the taking of views, and in particular and preferably:
   to specify to a user the number of images that they must acquire, and/or
   to guide a user when adjusting the geometry of the retractor support and/or positioning the mouth of the user on the dental retractor and/or positioning the dental retractor relative to the mouth of the user and/or the movements to be effected to acquire the images,
   a computer medium on which a program of this kind is stored, for example a memory or a CD-ROM, and
   a personal device, in particular a mobile telephone or a tablet, into which a program of this kind is loaded.

### Definitions

By “user” is meant any person for whom a dental retractor or a kit in accordance with the invention can be used, whether that person is ill or not, or whether that person is being treated or not. A retractor in accordance with the invention can be used for an animal other than a human being.

The service position is the position in which the retractor is disposed on the mouth of a user holding their head vertical, as depicted in FIG. 12.

The “mouth closed” position is the occlusion position in which the upper and lower teeth are in contact.

The “mouth open” position is the fully open position of the mouth.

The plane of the retractor is the plane of the opening that it defines.

A “tongue support” is conventionally a part of a dental retractor that penetrates to the interior of the mouth in such a manner as to interact with the tongue of the user in the service position, and in particular to serve it as a support and to move it away.

By “mobile telephone” is meant a device of less than 500 g weight, provided with a sensor enabling it to capture images, capable of exchanging data with another device more than 500 km from the mobile telephone, and capable of displaying said data, and in particular said images.

Two parts are “mobile elastically” relative to one another if their relative position may be modified by a force and if, when that force ceases, they return to their initial relative positions.

The right-hand and left-hand flanges are “opposed” flanges. The same applies to the upper and lower flanges. A dental retractor in accordance with the invention therefore includes two pairs each of which is formed of two opposite flanges, namely the right-hand and left-hand pair of flanges and the upper and lower pair of flanges.

The rigidity of the structure is not incompatible with the presence of mobile flanges, in particular flanges mobile elastically. In particular, the flanges are considered as being mobile. If the flanges are not mobile relative to one another then the retractor is totally rigid or has a locked rigidity. In particular, fixing the retractor support onto the dental retractor enables locked horizontal rigidity.

A “moving closer force” is a force exerted on a first flange, in the direction of the flange opposite said first flange, while the opposite flange is immobilized, in such a manner as to push the first flange toward the opposite edge.

The “acquisition conditions” for an image of the teeth specify the position and the orientation in space of an image acquisition device relative to the teeth of the user, and preferably the calibration of that image acquisition device to acquire said image at the moment of said acquisition by direct observation.

By “image” is meant a two-dimensional image such as a photograph. An image is formed of pixels.

Unless otherwise indicated the qualifiers used to define positions in space such as “horizontal”, “vertical”, “bottom”, “top”, “right-hand” or “left-hand” are defined, for clarity, with reference to a service position observed by a user having the head upright and their lips disposed on the dental retractor.

In the service position, the longitudinal axis X is the axis that extends perpendicularly to the general plane of the retractor opening and that passes through the center of the retractor opening.

“Fixing” one part to another means establishing a physical connection between these two parts, so that any movement of one of those parts ends up entraining that of the other part.

A part mounted to be mobile in translation, in rotation or in the manner of a ball joint on another part is fixed to that other part. Fixing may “definitive” or “removable”, depending on whether the two parts are adapted to be separated or not, preferably by hand, by a user.

Fixing may be rigid or non-rigid, according to whether it allows or does not allow relative movement of the two parts fixed to one another.

“Comprise”, “include” and “feature” must be interpreted broadly and not in a manner limiting on the invention unless otherwise indicated.

### BRIEF DESCRIPTION OF THE DRAWINGS

Other features and advantages of the invention will become more apparent on reading the following detailed description and examining the appended drawings, in which:

FIG. 1 represents a dental retractor in accordance with the invention, in perspective, represented from the side opposite the side intended to be positioned in the mouth of a user in the service position;

FIG. 2 represents the dental retractor from FIG. 1, in perspective, represented from the side intended to be positioned in the mouth of a user in the service position;

FIG. 3 represents the dental retractor from FIG. 1 represented as seen from above (3A), from the front (3B), from below (3C) and from the rear (3D);

FIG. 4 represents another dental retractor in accordance with the invention represented from the side intended to be positioned in the mouth of a user in the service position, and in perspective;

FIG. 5 is an example of a photograph of an occlusal view with a conventional dental retractor in the service position;

FIG. 6 is another example of a photograph of an occlusal view with a conventional dental retractor in the service position;

FIG. 7 is an example of a photograph of a front view including a dental retractor according to the invention in the service position;

FIG. 8 is an example of a photograph of a lateral view including a dental retractor according to the invention in the service position;

FIG. 9 is an example of a photograph of an occlusal view including a dental retractor according to the invention in the service position;

FIG. 10 represents, in perspective, a kit in accordance with the invention, the dental retractor being in the assembled position;

FIG. 11 represents, from a different perspective, a kit in accordance with the invention, the dental retractor being in the assembled position;

FIG. 12 represents the use of a kit in accordance with the invention in the service position;

FIG. 13 depicts the deformability of the structure of a retractor according to the invention;

FIG. 14 depicts the deformability of the upper flange of a retractor in accordance with the invention;

FIG. 15 represents, in the left-hand part, in perspective, a retractor support of a kit in accordance with the invention and, in the right-hand part, a section depicting the attachment of the dental retractor to said retractor support;

FIG. 16 depicts the deformability of upper and lower flanges and

FIG. 17 represents a retractor that has been subjected to comparative tests.

In the various figures identical or analogous members have been designated using identical references.

### DETAILED DESCRIPTION

Dental Retractor

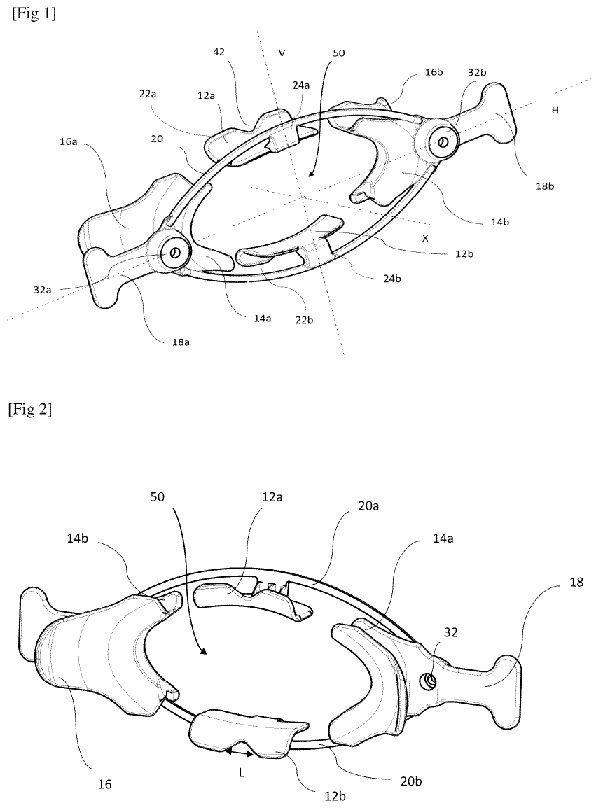

The dental retractor 10 represented in FIGS. 1, 2 and 3 is symmetrical with respect to a median plane which, in the service position, extends vertically (the vertical and horizontal directions are denoted “V” and “H”, respectively).

It includes a right-hand flange 14a (on the user's right in the service position), a left-hand flange 14b, an upper flange 12a, a lower flange 12b, and a structure 20 surrounding an opening 50 in the retractor including, in the example represented, an upper arch 20a and a lower arch 20b.

The opening in the retractor is entirely free, that is to say that when the retractor is observed along the axis X no part of the retractor blocks even partially the opening in the retractor. The dental retractor is thus configured in such a manner as to leave entirely unobstructed the view through the retractor opening in the service position, which advantageously prevents any concealment of a tooth.

In particular, the dental retractor does not have any crossmember, for example a horizontal crossmember joining the right-hand and left-hand flanges or the right-hand and left-hand ends of the structure, partially blocking said view. In particular, the dental retractor preferably does not include a tongue support. The retractor may advantageously be deformed more easily to be placed in the service position.

The retractor preferably includes no part extending, in the service position, from the side of the intrados of the teeth. In other words, in the service position and in the closed position of the mouth the retractor does not penetrate into the buccal cavity partly delimited by the intrados of the teeth. In the service position it extends exclusively outside the mouth and in the space defined by the extrados of the teeth and the lips. The user is therefore able to close the mouth to the point of occlusion.

Said right-hand flange 14a, left-hand flange 14b, upper flange 12a and lower flange 12b are fixed to said structure 20 on the right, on the left, above and below said retractor opening 50, respectively, right and left being defined relative to a situation in which the dental retractor is observed from the side intended to be positioned in the mouth of the user.

To be more precise, in the example represented in FIGS. 1 to 3 the upper flange 12a and the lower flange 12b are fixed to the middle of the upper arch 20a and the lower arch 20b, respectively.

The right-hand ends of the upper arch 20a and the lower arch 20b join at a right-hand junction. The left-hand ends of the upper arch 20a and the lower arch 20b join at a left-hand junction. The right-hand and left-hand junctions therefore define the right-hand and left-hand ends of the structure, respectively.

The arches together form a flattened ring the shape of which is similar to the contour of a rugby ball. The slope a of the arches relative to a horizontal plane is preferably maximum at the level of the right-hand and left-hand junctions and preferably zero substantially in the middle of the arches.

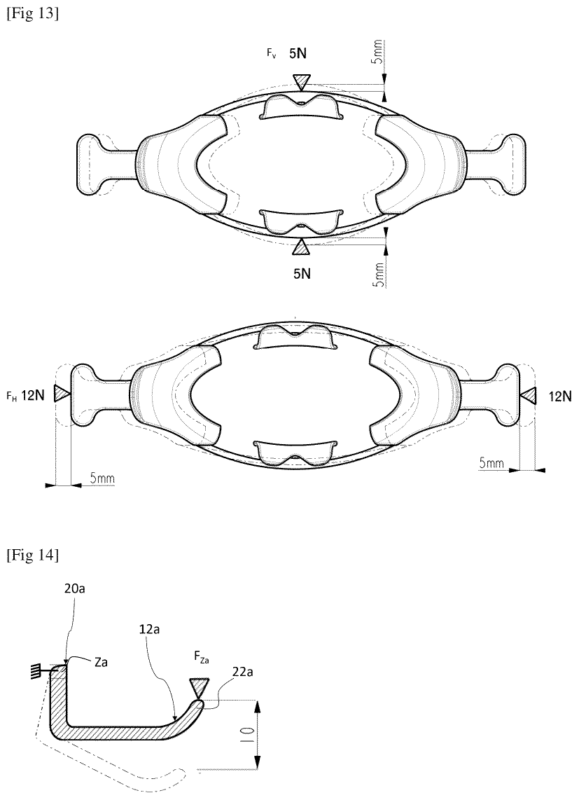

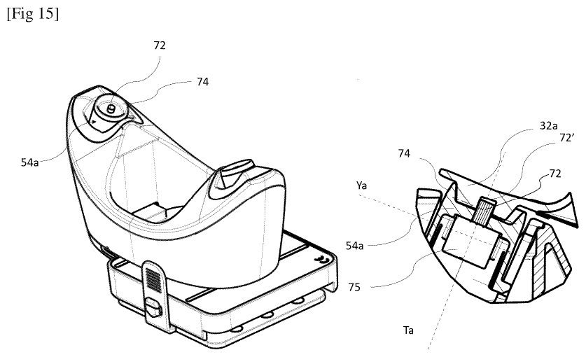

As depicted in FIG. 13 the retractor can be deformed by a vertical force F_{V} or a horizontal force F_{H}. In FIG. 13 the forces F_{V} and F_{H} are exerted at the middle of the upper arch and at the left-hand junction between the arches, respectively, and are therefore forces for moving closer together “the arches” closer together.

Generally speaking the structure is relatively rigid horizontally. It can preferably be compressed horizontally by 10 mm only by a horizontal force F_{H} moving closer together the two lateral ends (right-hand and left-hand ends) of the structure, greater than 5 N, preferably greater than or equal to 6 N, preferably greater than or equal to 8 N, preferably greater than 10 N.

In the embodiment depicted the horizontal force F_{H} for moving the right-hand and left-hand flanges horizontally 10 mm closer together by acting exclusively on the junctions of the arches, is preferably less than 20 N, 15 N and/or greater than 5 N, preferably greater than or equal to 6 N, preferably greater than or equal to 8 N, preferably greater than or equal to 10 N. In FIG. 13 it is 12 N.

The vertical force F_{V} for moving the upper and lower flanges 10 mm closer together horizontally by acting exclusively in the middle of the arches is less than 15 N, 13 N, 10 N, 8 N, preferably less than or equal to 5 N, and/or greater than 3 N, preferably greater than 4 N. In FIG. 13 it is 5 N.

The upper flange 12a and the lower flange 12b may be rigidly fixed to the structure 20. As represented in FIG. 14, the upper flange 12a and/or the lower flange 12b, preferably each of the upper flange 12a and the lower flange 12b, is fixed to the upper arch or to the lower arch, respectively, in such a manner as to be able to move relative to the upper arch and to the lower arch, respectively, preferably by pivoting elastically, about an “upper” or “lower” axis Zₐ, respectively, which is horizontal in the service position. This improves comfort.

This elasticity preferably enables the upper free edge 22a of the upper flange 12a or the lower free edge 22b of the lower flange 12b, respectively, be moved vertically by a distance of 10 mm by a force (referenced F_{Za} in FIG. 14) less than 10 N, 5 N, 3 N, 2 N, preferably less than or equal to 1.5 N, and/or greater than 0.5 N, preferably greater than 1 N. To measure this movement the structure is kept rigid. The force F_{Za} therefore results exclusively in movement, preferably pivoting, of the upper flange 12a or of the lower flange 12b, respectively (FIG. 14).

In a preferred embodiment the upper junction 24a and the lower junction 24b of the upper flange 12a and the lower flange 12b form pivot connections, preferably about a horizontal axis in the service position. The pivoting of the upper flange 12a and the lower flange 12b advantageously enables the lips to be moved closer together when the use closes their mouth.

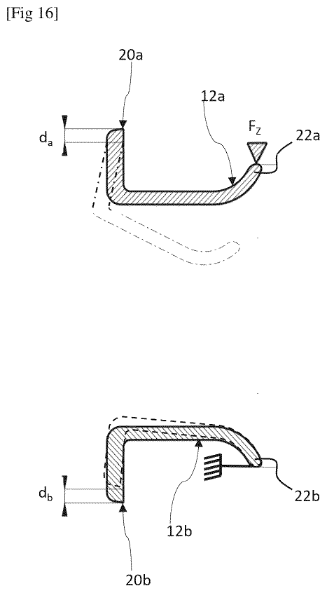

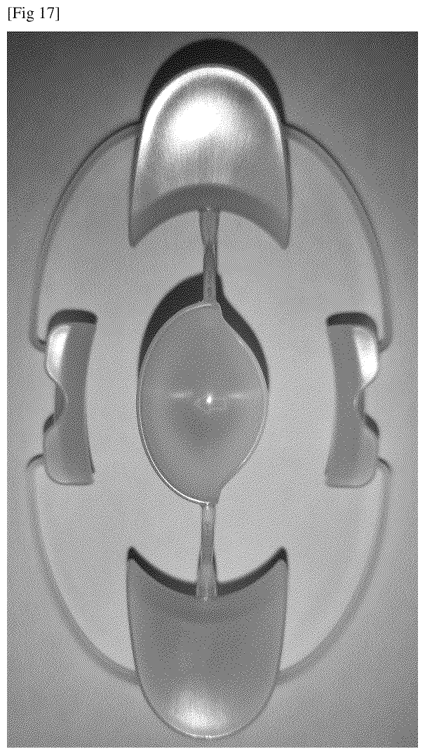

In one embodiment, as depicted in FIG. 16, the structure is also deformable in such a manner that, when the retractor is not fixed to the support, pinching to move vertically 20 mm closer together the upper free edge 22a and the lower free edge 22b also produces vertical movement closer together of the upper and lower arches by a distance d (=dₐ+d_{b}). The vertical force for moving the upper and lower flanges vertically 10 mm closer together, by acting exclusively on the free edges of said upper and lower flanges, is preferably less than 8 N, 6 N, 4 N, 3 N, 2 N, preferably less than or equal to 1.5 N, and/or greater than 0.5 N, preferably greater than 1 N. In the preferred embodiment this movement closer together therefore takes account of the flexibility of the arches but also of the pivoting of the flanges on the arches. The vertical force for movement closer together may be exerted on the free edge of the upper flange, the free edge of the lower flange being kept immobile and the dental retractor being held only by the free edge of the lower flange. Tests have shown that with a retractor of the type described in U.S. Pat. No. 6,923,761 or WO2006014371 cited in the preamble, marketed by the company YDM and represented in FIG. 17, a vertical closer together force of 0.6 N is sufficient to move the free edges of the upper and lower flanges 10 mm closer together.

The right-hand flange 14a and left-hand flange 14b (lateral flanges) are fixed, preferably rigidly, to the right hand and left-hand junctions of the upper and lower arches.

The structure 20 may constitute a single piece, having a substantially ellipsoidal shape for example.

The structure 20, surrounding the retractor opening 50, advantageously includes an opening larger than the retractor opening 50. Thus the structure 20 does not obstruct the retractor opening in the service position in which the structure 20 is outside the mouth of the user and extends around the mouth.

As represented in the example from FIGS. 1 to 3 the right-hand, left-hand, upper and lower flanges are preferably made in one piece with the retractor structure.

Each flange preferably takes the form of a trough intended to support part of the lips of the user when the dental retractor is in the service position.

Each flange 12 has a length L₁₂ or L₁₄ measured along the bottom of the trough greater than 1 cm, preferably greater than 2 cm, preferably greater than 3 cm and/or less than 15 cm, preferably less than 12 cm, preferably less than 8 cm.

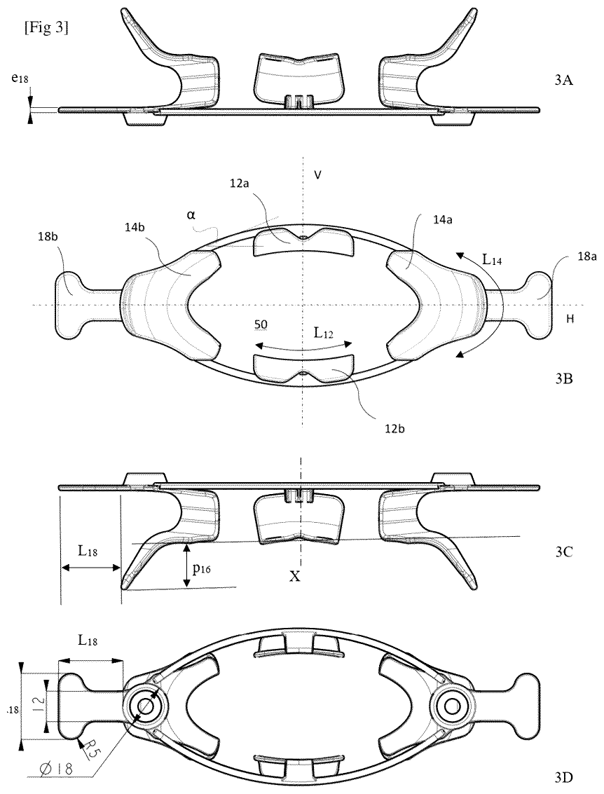

The upper and lower flanges are preferably the same length L₁₂. The right-hand and left-hand flanges are preferably the same length L₁₄. The length L₁₄ of the lateral flanges is preferably greater than the length L₁₂ of the upper and lower flanges (FIG. 3).

The length L₁₂ is preferably greater than 1 cm, preferably greater than 2 cm, preferably greater than 2.5 cm, preferably greater than 3 cm, and/or less than 5 cm, preferably less than 4 cm.

The length L₁₄ is preferably greater than 3 cm, preferably greater than 4 cm, and/or less than 9 cm, preferably less than 8 cm.

In the FIG. 1 example the upper flange 12a and the lower flange 12b each include a notch 42 on the free edge of the part of the flange intended to be introduced into the mouth of the user. This notch preferably has a triangular shape with a maximum width L between 3 mm and 15 mm inclusive, better still between 5 mm and 10 mm inclusive. Alternatively, the notch may have a rounded or rectangular shape.

The notch 42 is situated in the middle of the flange that carries it and enables the comfort of the user to be improved when the dental retractor is in the service position. In fact, in the service position the notch makes it possible to leave free a labial brake at the level of the upper and lower dental arches. The amplitude of the notch further enables movement of the dental retractor in the mouth of the user, in particular rotation about the dental arches to acquire lateral view photos.

In the embodiment represented the retractor also includes a right-hand lug 16a and a left-hand lug 16b adapted to move the cheeks away from the teeth. In this example the lugs 16 have a concave shape facing the axis X, advantageously adapting to the shape of the cheeks. Said lugs 16 formed in this way enable further improvement of the comfort of the dental retractor for the user.

The right-hand lug 16a and the left-hand lug 16b are preferably of identical shape. They are preferably conformed in such a manner as to extend along the axis X beyond the region in which the flanges extend, over a depth p₁₆ greater than 1 cm, preferably greater than 1.5 cm, and/or less than 5 cm, preferably less than 4 cm, preferably less than 3 cm, preferably less than 2.5 cm (see FIG. 3).

The edge of the lugs is preferably rounded, in such a manner as to be atraumatic.

In the embodiment represented the retractor further includes a right-hand handle 18a and a left-hand handle 18b preferably extending substantially parallel to the general plane of the retractor perpendicular to the retractor opening axis X. The right-hand handle 18a and the left-handle 18b advantageously facilitate manipulation of the dental retractor, in particular when placing it in the service position.

The right-hand handle 18a and the left-handle 18b are preferably the same shape. They preferably take the form of tabs, preferably substantially flat tabs, that extend to the right and to the left of the right-hand and left-hand junctions of the upper and lower arches, respectively.

The right-hand handle 18a and the left-hand 18b are preferably oriented toward the center of the extractor opening.

They preferably extend substantially horizontally in the service position.

They preferably extend substantially in a plane parallel to the plane of the retractor opening, preferably in the plane of the arches.

Each handle has:
a length L₁₈ preferably greater than 1 cm, preferably greater than 1.5 cm, preferably greater than 2 cm, preferably greater than 2.5 cm and/or less than 10 cm, preferably less than 8 cm, preferably less than 6 cm, preferably less than 4 cm, preferably less than 3 cm, and/or
a maximum width l₁₈, preferably at the free end of the handle, preferably greater than 1 cm, preferably greater than 1.5 cm, preferably greater than 2 cm, preferably greater than 2.5 cm and/or less than 10 cm, preferably less than 8 cm, preferably less than 6 cm, preferably less than 4 cm, preferably less than 3 cm, and/or
a thickness e₁₈ preferably greater than 0.1 cm, preferably greater than 0.15 cm and/or less than 0.8 cm, preferably less than 0.5 cm, preferably less than 0.4 cm, preferably less than 0.3 cm.

The length L₁₈ of a handle is measured from the structure of the retractor.

In FIG. 3 the handle extends horizontally and its length is also the distance over which it extends horizontally from the end of the closest lateral flange when the retractor is observed along its axis X.

The right-hand and left-hand handles are preferably fixed, preferably rigidly fixed, to the right-hand and left-hand ends of the structure and/or to the right-hand and left-hand flanges.

In a preferred embodiment the right-hand and left-hand handles are made in one piece with the right-hand and left-hand flanges, respectively, in the form of monobloc right-hand and left-hand junction pieces, respectively, to which the right-hand ends and the left-hand ends of the arches, respectively, are fixed, preferably rigidly fixed (see FIG. 1).

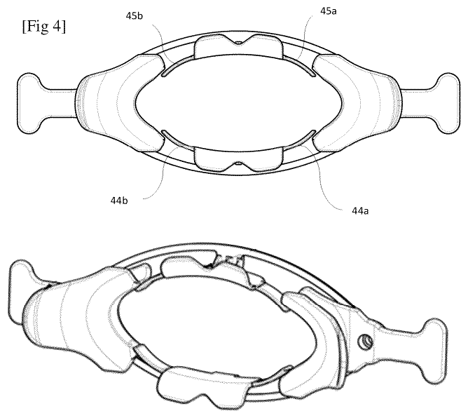

The right-hand and left-hand handles are shaped so that the user can position the dental retractor in said service position without their fingers coming into contact with their mouth. The right-hand and left-hand handles are preferably arranged in such a manner as to extend radially to the outside of the retractor opening, preferably in such a manner as not to be in contact with the skin of the user, and in particular with their cheeks, in the service position. The right-hand and left-hand handles therefore enable good hygiene to be preserved and are therefore particularly suitable if the dental retractor is liable to be put into the service position by the user himself or herself. In a preferred embodiment the dental retractor also includes, as represented in FIG. 4, right-hand lower fins 44a and left-hand lower fins 44b and right-hand upper fins 45a and left-hand upper fins 45b.

The lower fins 44a; 44b and upper fins 45a; 45b are respectively fixed to the lower fin 12b and the upper fin 12a and the fins are preferably made in one piece with the flanges.

Because of deformation of the dental retractor, in particular in torsion about a horizontal axis or flexing about a vertical axis of the dental retractor structure, the right-hand lower and upper fins 44a; 45a come to abut against the right-hand flange 14a and the left-hand lower and upper fins 44b; 45b come to abut against the left-hand flange 14b. This advantageously limits the movement farther apart of the upper and lower flanges.

Kit

In one embodiment the dental retractor includes retractor attachment parts 32 on the right and on the left of the retractor opening. The retractor attachment parts 32 are intended to cooperate with corresponding support attachment parts.

As represented in FIG. 1 these retractor attachment parts may be magnetic inserts 32a; 32b for fixing the retractor support to the dental retractor. These inserts 32a and 32b are carried by the right-hand flange 14a and the left flange 14b, respectively.

The support attachment parts and the retractor attachment parts 32, and in particular the magnet(s) and metal part(s), are preferably configured in such a manner that the dental retractor can be fixed to the retractor support only in a unique predetermined position.

Thus it advantageously suffices for the user to move the retractor support closer to the dental retractor for the dental retractor to become fixed to the retractor support. Using magnetic fixing also enables reliable and very precise fixing, even if the user is not looking at the retractor support or the dental retractor. When the dental retractor is in the service position the user can therefore easily position the retractor support on the dental retractor, precisely and without any training.

The dental retractor is made at least in part of elastomer.

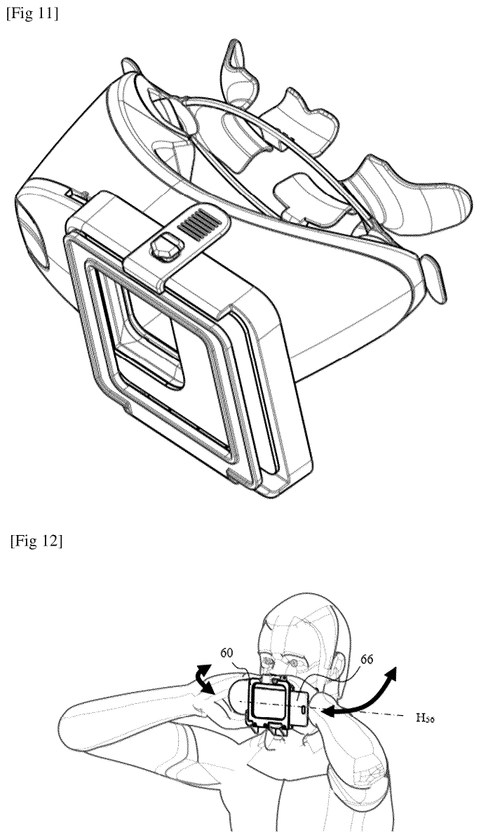

The retractor support 52, represented in FIGS. 10 and 11, preferably has the general shape described in the application EP 3 391 810, preferably the shape of the support 12 described in WO2020/089248. Alternatively, the retractor support may take the form of a connecting shaft or a tube, for example an arm connecting the image acquisition device to the dental retractor.

The retractor support 52 preferably includes a body 53, a right-hand support attachment part 54a and a left-hand support attachment part 54b rigidly fixed to the body 53, preferably magnets intended to cooperate with the right-hand attachment part 32a and the left-hand attachment part 32b of the retractor, respectively, and a support for the image acquisition device, preferably a mobile telephone support 56. The adjustable or constant distance A between the right-hand and left-hand support attachment parts, which is preferably constant, determines the distance between the right-hand flange 14a and the left-hand flange 14b in the assembled position. It therefore determines the stiffness of the structure of the retractor in the assembled position. As described hereinabove, the free edges of the upper and lower flanges may preferably move closer together elastically, by pivoting about respective horizontal axes.

Each support attachment part is preferably articulated on the body 53. This articulation makes it possible to facilitate modification of the regions of the mouth observed by the camera of the mobile telephone through the retractor opening. This facilitates acquisition of photos by the user.

An articulation is a connection allowing rotation about at least one rotation axis.

In a preferred embodiment each support attachment part is mounted to pivot on the body 53. The articulation is therefore a pivot connection, preferably about a pivot axis, preferably horizontal in the service position. It then allows only rotation about that axis. At least in extreme bottom and top pivoting positions, pivoting preferably allows photos to be taken of the palate and of the tongue, respectively.

The pivot axes of the right-hand support attachment part 54a and the left-hand support attachment part 54b are referenced Ya et Yb, respectively.

The pivot axis may be fixed relative to the retractor support.

The pivot axis Ya and/or Yb is (are) preferably mobile in translation relative to the body 53 in a direction different from that of the pivot axis, preferably along a horizontal axis Ta and/or Tb, preferably along an axis perpendicular to the pivot axis. The maximum amplitude of the movement in translation is preferably 5 mm, 8 mm, 10 mm or 12 mm and/or less than 25 mm, 20 mm, 18 mm or 16 mm.

When the axis of movement in translation is not perpendicular to the pivot axis the pivot connection is replaced by a ball-joint connection.

In the preferred embodiment of the invention, in the mounted position of the retractor, each retractor attachment part is fixed magnetically and removably to a support attachment part. The axes Ta and Tb are the axes along which the retractor and support attachment parts are attracted toward each other, and then pressed one against the other. They are referred to as “attachment axes”.

As depicted in FIG. 15 said retractor and support attachment parts of an attachment are configured to block, by abutment, movement, preferably all movement, in a plane perpendicular to the attachment axis of that attachment.

Each attachment preferably includes a plurality of abutments enabling such blocking.

In the FIG. 15 embodiment each attachment includes two abutments enabling such blocking, in the form of a pin 72 and a circular flange 74.

The pin 72 represented is part of the support, and to be more precise is rigidly fixed to a shaft 75 rotatably mounted on the support to rotate about the pivot axis Ya of the right-hand support attachment part 54a. It extends along the attachment axis Ta and projects from the support in such a manner as to be able, in the assembled position, to be housed in a housing 72′ of complementary shape in the retractor attachment part 32a. The cooperation of the pin and of the housing enables an abutment to be provided if a detachment force is exerted perpendicularly to the attachment axis Ta, in particular if the user has placed the retractor in their mouth and pulls the support toward the right, toward the left, upward or downward to acquire lateral or occlusal views.

The pin 72 may be any shape. It is preferably cylindrical, preferably of circular section. It is preferably shaped to penetrate into the housing over a distance greater than 1 mm, preferably greater than 2 mm and/or less than 15 mm, preferably less than 10 mm, preferably less than 7 mm.

The pin may equally well be frustoconical in order to facilitate its insertion in the housing. The flange 74 represented is part of the support, and to be more precise is rigidly fixed to the shaft 75. It is circular about an axis Ta and projects from the support in such a manner as to define a housing able, in the assembled position, to receive the retractor attachment part 32a.

The housing may equally be flared, as represented here, in order to facilitate the insertion of the retractor attachment part 32a.

The flange 74 and the retractor attachment part 32a may be complementary. In the embodiment represented the flange 74 defines however a housing slightly larger than the retractor attachment part 32a that is receives. The flange 74 is therefore advantageously able to guide the user when they assemble the retractor and the support, in particular to facilitate the introduction of the pin 72 in the housing 72′.

The preferably constant height of the flange is preferably greater than 1 mm, preferably greater than 2 mm and/or less than 15 mm, preferably less than 10 mm, preferably less than 7 mm.

The cooperation of the flange 74 and of the retractor attachment part 32a also makes it possible to provide an abutment when a detachment force is exerted perpendicularly to the attachment Ta, in particular when the user has placed the retractor in their mouth and pulls the support toward the right to acquire photos seen from the right.

The axes of the flange 74 and of the pin 72 are parallel and preferably coincide.

Of course other abutment configurations are possible. In particular, the pin 72 and/or the flange 74 could be part of the retractor attachment part. The abutments do not necessarily provide blocking in all directions parallel to a plane perpendicular to the attachment axis. The flange may be interrupted, for example.

The mobile telephone support 56 preferably includes a base 58 and a door 60 mounted to pivot on the base 58 and shaped in such a manner that a mobile telephone, not represented, can be laid flat on the base 58 and then clamped between the base 58 and the door 60. The door 60 includes a flexible locking strap 62 that can be selectively hooked onto a pin 64 of the base 58 in such a manner as to retain the mobile telephone 66 in this clamped position, as represented in FIG. 12.

Any known means may be used for fixing the mobile telephone, for example an elastic band, self-gripping bands, clips, screws, a flap, for example mounted to rotate on the support, lockable in a position in which it clamps the image acquisition device, or a sucker. This list of examples is not limiting on the invention.

The base is preferably mounted to be mobile on the body 53, preferably mounted to be mobile in translation, preferably mobile in translation exclusively in a horizontal direction H₅₆, in such a manner as to be able to move the optical axis of the mobile telephone relative to the retractor opening 50.

The overall rigidity of the structure of the dental retractor in the assembled position, resulting from the distance A defined between the support attachments, in particular enables the user to move the dental retractor around the dental arch of the user in order to acquire photos of different views, for example modifying horizontally and/or vertically the position of the dental retractor, preferably when holding the retractor by the handles 18. Thus the user can easily acquire lateral and/or occlusal images of the teeth of the user by means of the image acquisition device fixed to the retractor support and positioned facing the retractor opening.

In one particular embodiment the image acquisition device is integrated into the retractor support. The image acquisition device is preferably a mobile telephone.

Method

Before step a), in the case of using a kit according to the invention, the image acquisition device, preferably a mobile telephone, is fixed to the retractor support, preferably in a removable manner.

In particular when the retractor support defines a closed chamber between the mobile telephone and the retractor, the mobile telephone is preferably fixed to the retractor support so that the screen of the mobile telephone is oriented on the side opposite the retractor. Thus in the service position the user can advantageously view this screen, for example in a mirror.

When the retractor support enables the user to view the mobile telephone in the service position directly the mobile telephone is fixed so that the rear camera of the mobile telephone faces the user in the service position. The user can therefore, in the service position, advantageously view the screen of the mobile telephone.

In step a) the user, preferably a patient undergoing orthodontic treatment, positions a dental retractor as described above in their mouth.

In a preferred embodiment the user moves the right-hand handle 18a and the left-hand handle 18b closer together by twisting the dental retractor and then introduces the dental retractor into their mouth, keeping it bent. The user preferably deforms the structure by bending the rigid structure about a vertical axis passing through the center of the retractor opening. The optional fins 44a; 44b; 45a; 45b make it possible to limit the distance between the upper flange 12a and the lower flange 12b when the handles 18 are moved closer together. The user can then easily introduce the dental retractor into their mouth and then let go of the handles 18a and 18b so that they return elastically into a position close to their initial position, better still into their initial position, which is preferably substantially perpendicular to the axis X, as represented in FIG. 4 for example. The user can equally impart a twist about a horizontal axis. The user then first positions a lateral flange (right-hand flange or left-hand flange) on the corresponding commissure, for example the right-hand flange on the right-hand commissure, and then positions the opposite flange on the opposite commissure, for example the left-hand flange on the left-hand commissure, whilst maintaining the twist during positioning.

Alternatively, the user first introduces into their mouth a first of the right-hand and left-hand flanges and then deforms the dental retractor so as to introduce therein the second of the right-hand and left-hand flanges.

The deformability of a dental retractor according to the invention enables easier insertion in the mouth of the user.

In the case of using a kit according to the invention, in step b) the user fixes the support attachment parts onto the retractor attachment parts. In a preferred embodiment the retractor and retractor support attachment parts together form a magnet-based system facilitating fixing of the retractor support onto the dental retractor.

Alternatively or additionally, the retractor and retractor support attachment parts include at least one elastic band intended to hold the retractor support against the dental retractor. For example, an elastic band may be retained on the structure 20 of the dental retractor, at the level of the lateral (right-hand and left-hand) flanges, and the retractor support may include hooks or a groove intended to receive the elastic band and facilitating positioning of the retractor support relative to the retractor. Alternatively and/or additionally, the user may be guided to fix the dental retractor to the retractor support. The user is preferably guided by means of visual and/or audio instructions, for example emitted by the mobile telephone via a mobile application.

The instructions are for example displayed on the screen of the mobile telephone.

The instructions may include:
schematics depicting various steps to be taken, for example depicting the various steps of the method,
visual indicators such as arrows explaining the movements to be performed by the user to acquire lateral or occlusal views, for example,
marker points enabling positioning of the dental retractor relative to the user or the dental retractor relative to the retractor support,
tooth models indicating to the user the content of the image to be acquired and facilitating positioning of the mobile telephone relative to the user, the user being able for example to superpose approximately the displayed toothed model and the preview of the teeth of the user appearing on the screen before acquiring an image.

Alternatively the instructions may be on a paper medium.

In step c), by opening or closing the jaws the user can modify in particular the angle of acquisition by rotation of the acquisition kit around the head of the user, while maintaining the dental retractor in the service position, the distance separating the image acquisition device and the teeth of the user, and/or the relative position of the jaws.

For example, from a central initial position the user turns the retractor support and the dental retractor around the head of the user by an angle of more than 15°, 20°, 30°, 45° (arrows in FIG. 12), in such a manner that the right-hand molars face the retractor opening, and then in such a manner that the left-hand molars face the retractor opening. The user can easily acquire an image of the molars thanks to the rigidity of the dental retractor in the assembled position enabling separation of the cheeks and lips without the dental retractor being deformed by said cheeks and/or said lips. In the same way, a dental retractor of this kind enables easy acquisition of occlusal views.

The user can equally open or close their mouth.

In step d) the user acquires one or more images by pressing the trigger of the acquisition device. Alternatively, acquisition is effected automatically when acquisition conditions are complied with or at the end of a pre-defined time, for example every 10 s, every 5 s.

In a preferred embodiment the user acquires a plurality of images, preferably at least three images, preferably at least five images.

The acquisition conditions are preferably modified between image acquisitions. In particular, the user preferably acquires jaws closed views, jaws open views, lateral views, occlusal views and/or front views.

The pin 72 and the flange 74 advantageously provide an abutment limiting the movement of the support relative to the retractor when the user pulls the support laterally or vertically to acquire images from their right or from their left or occlusal images. They therefore limit the risk of detachment of the support from the retractor during these operations. However, they do not oppose pulling along the attachment axes Ta and Tb. The user can therefore easily detach the support and the retractor after use by exerting forces tending to move them farther apart along those axes.

### Examples

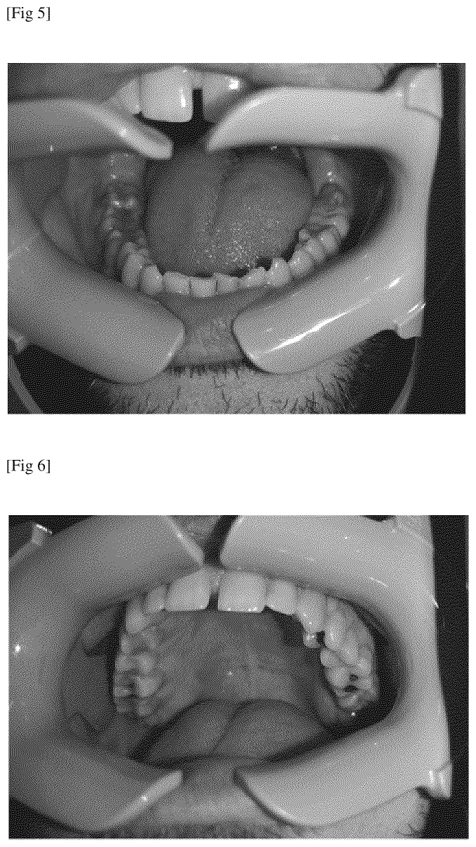

FIGS. 5 and 6 are occlusal view photos resulting from acquisition of dental arch images using a dental retractor conventionally used in the prior art.

In contrast to the retractor according to the invention the retractor from FIGS. 5 and 6 does not move the lips completely away from the teeth. In particular, it can be seen in FIG. 5 that the bottom lip of the user conceals some of the teeth, in particular the incisors.

In the FIG. 6 example the retractor itself conceals some of the teeth.

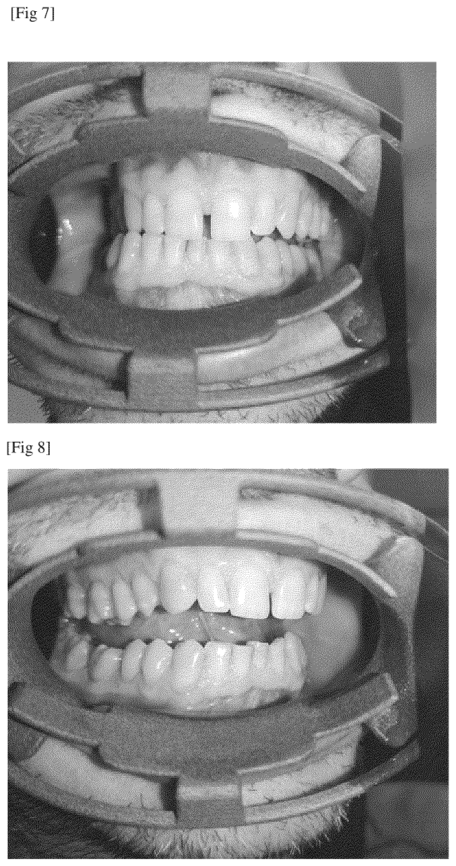

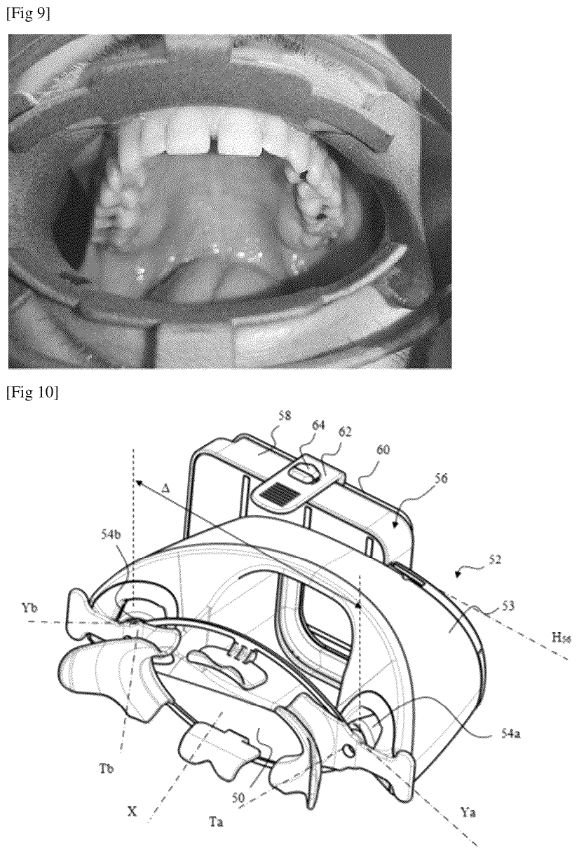

In contrast, as can be seen in FIGS. 7 to 9, the teeth are well exposed and visible during acquisition of the images by means of a dental retractor according to the invention. In particular, FIG. 9 depicts an occlusal view like FIG. 6.

As is now clearly apparent, a dental retractor according to the invention has the following benefits:
relative deformability of the retractor for positioning it in the mouth of the user, then
stiffness of the structure, possibly reinforced by attachment to a support, which facilitates observation of the teeth at the back of the mouth, and
vertical deformability, facilitating closing of the mouth.

This retractor is advantageously simple, easy to use and robust, and likewise the kit.

Of course, the invention is not limited to the embodiments described hereinabove and represented.

In particular, the magnetic fixing may be achieved with two magnets or with a magnet and a metal insert.

1. A dental retractor including:
a right-hand flange, a left-hand flange, an upper flange and a lower flange extending around a retractor opening and intended to bear, in a service position, on the right-hand labial commissure, the left-hand labial commissure, the central part of the upper lip and the central part of the lower lip of a user, respectively,
a structure onto which said flanges are fixed, and
right-hand and left-hand handles rigidly fixed to right-hand and left-hand ends of the structure, respectively, and/or to the right-hand and left-hand flanges, respectively, shaped in such a manner as to extend to outside the mouth of the user in the service position,
in which retractor the structure and/or the fixing of the flanges onto the structure is/are shaped in such a manner that the horizontal moving closer force necessary to move the right-hand flange and the left-flange 10 mm closer together is greater than 5 N,
a moving closer force between two flanges being a traction force exerted, parallel to the plane of the retractor, on the free end of one of the two flanges, to move it elastically closer to the free end of the other flange, which is kept immobile, the horizontal and vertical directions being defined in the service position in which the retractor is disposed on the mouth of the user, who is maintaining their head vertical.

2. The dental retractor as claimed in claim 1, in which the horizontal moving closer force is greater than or equal to 10 N.

3. The dental retractor as claimed in claim 1, in which the structure is compressible horizontally by 10 mm only by the action of a force greater than 5 N exerted horizontally, parallel to the plane of the retractor, on a lateral end of said structure to move it elastically closer to the other lateral end of said structure, which is kept immobile.

4. The dental retractor as claimed in claim 3, in which said force is greater than or equal to 10 N.

5. The dental retractor as claimed in claim 1, in which the right-hand and left-hand handles are arranged in such a manner as to extend radially to the outside of the retractor opening in the service position.

6. The dental retractor as claimed in claim 1, configured in such a manner as to leave entirely unobstructed the view through the retractor opening in the service position.

7. The dental retractor as claimed in claim 1, in which the structure is shaped in such a manner as to extend radially outside the mouth of the user in the service position.

8. The dental retractor as claimed in claim 1, shaped in such a manner as to extend, in the service position, only outside the mouth of the user and in the space defined by the extrados of the teeth and the lips of the user.

9. The retractor as claimed in claim 1, the upper flange and the lower flange being fixed to the structure of the retractor by junctions allowing vertical elastic movement of the free edges of said flanges relative to said structure, the amplitude of said movement being greater than 10 mm.

10. The dental retractor as claimed in claim 9, in which said junctions allow said elastic movement because of the effect of a force less than 10 N and greater than 0.5 N exerted on the upper free edge of the upper flange or on the lower free edge of the lower flange.

11. The dental retractor as claimed in claim 1, in which the structure includes upper and lower arches joined at their ends in such a manner that the structure has an oval overall shape.

12. The dental retractor as claimed in claim 11, in which at any point on said upper and/or lower arches the slope of the arch is greater than or equal to 0° and less than or equal to 60°, the slope at a point of the arch being the absolute value of the smallest angle formed by a tangent to said arch at said point with a horizontal plane in the service position.

13. The dental retractor as claimed in claim 11, in which the upper and lower arches are arranged in such a manner as to extend radially outside the retractor opening in the service position.

14. The dental retractor as claimed in claim 11, the upper and lower arches being configured to be moved closer by more than 10 mm by a vertical force moving said arches closer together that is lower than 15 N, the force moving the two arches closer together being a traction force exerted parallel to the plane of the retractor at the middle of one of said arches to move it elastically closer to the middle of the other arch, which is kept immobile.

15. The dental retractor as claimed in claim 11 the upper and lower flanges being fixed to an upper arch and a lower arch of the structure of the retractor, respectively, the upper flange and/or the lower flange being mounted to pivot elastically on the upper and lower arches, respectively, said pivoting of the upper flange and/or of the lower flange allowing vertical movement of the free edge of the upper flange and/or of the lower flange, respectively, by a distance of 10 mm because of the effect of a force less than 10 N.

16. The dental retractor as claimed in claim 11, in which the right-hand and left-hand flanges are fixed rigidly to right-hand and left-hand ends, respectively, of the upper and lower arches.

17. The dental retractor as claimed in claim 1, the upper flange and/or the lower flange including a right-hand fin and a left-hand fin configured to come into abutment with the right-hand flange and the left-hand flange, respectively, in such a manner as to impede movement apart of the upper flange and the lower flange during movement closer together of the right-hand and left-hand flanges.

18. The dental retractor as claimed in claim 1, the upper flange and/or the lower flange including a notch to receive a labial brake.

19. The dental retractor as claimed in claim 1, the color of at least a part of the retractor having, in RGB code, substantially the same value for red, green and blue, the maximum difference between the values for red, green and blue being less than 10.

20. The dental retractor as claimed in claim 1, in which the structure and/or the fixing of the flanges onto the structure is/are shaped in such a manner that the “vertical” moving closer force necessary to move the upper flange and the lower flange 10 mm closer together is less than the “horizontal” moving closer force necessary to move the right-hand flange and the left-hand flange 10 mm closer together.

21. The dental retractor as claimed in claim 20, the ratio of the horizontal moving closer force to the vertical moving closer force being greater than 5.

22. The dental retractor as claimed in claim 20 in which said horizontal moving closer force is less than 20 N and said vertical moving closer force is less than 8 N.

23. The dental retractor as claimed in claim 1, in which the retractor opening is entirely unobstructed in such a manner that when the dental retractor is observed along the axis X of the retractor opening no part of the retractor conceals the retractor opening even partially.

24. A kit including:
a dental retractor as claimed in claim 1 and including retractor attachment parts, and
a retractor support including support attachment parts disposed in such a manner as to cooperate, preferably magnetically, with said retractor attachment parts in such a manner as, in an assembled position of the retractor on the retractor support, to impose a distance between the right-hand flange and the left-hand flange.

25. The kit as claimed in claim 24 in which in the assembled position each retractor attachment part is pressed onto a corresponding support attachment part along an attachment axis, said retractor and support attachment parts being configured to block by means of abutment movement in a plane perpendicular to the attachment axis.

26. The kit as claimed in claim 24 including a mobile telephone removably fixed onto the retractor support in a position enabling said image acquisition device to acquire an image representing the retractor opening.

27. A method of acquiring dental images by means of a dental retractor as claimed in claim 1, said method including the following steps:
a) placing the dental retractor in the service position,
b) if the method is executed by means of a kit, after step a), fixing the retractor support to the dental retractor so that the dental retractor is in the assembled position,
c) moving the dental retractor by causing the dental retractor to perform a movement in rotation around the user, toward the right and/or toward the left of the user, so as to render at least one molar visible through the retractor opening, and/or closing the mouth of the user so as to render teeth visible through the retractor opening in the mouth closed position,
d) acquiring by means of an image acquisition device at least one image representing said molar and/or representing teeth in the mouth closed position.

28. The method as claimed in claim 27 in which, before step d), the image acquisition device is fixed to the retractor support in a position enabling said image acquisition device to acquire an image representing the retractor opening.
